SEQUENCE LISTING (5 sequences)

SEQ ID NO: 1 - DNA, 1771 bp, Homo sapiens
gcagccggctccctgaggcccaggaaccaccgcggcggccggcaccacctggacccagctccaggcgggcgcggcttcagcaccacggacagcgccccacccgcggccctccccccggcggcgcgctccagccggtgtaggcgaggcagcgagctatgcccgcggcatggcccggcgctgcggccccgtggcgctgctccttggcttcggcctcctccggctgtgctcaggggtgtggggtacggatacagaggagcggctggtggagcatctcctggatccttcccgctacaacaagcttatccgcccagccaccaatggctctgagctggtgacagtacagcttatggtgtcactggcccagctcatcagtgtgcatgagcgggagcagatcatgaccaccaatgtctggctgacccaggagtgggaagattatcgcctcacctggaagcctgaagagtttgacaacatgaagaaagttcggctcccttccaaacacatctggctcccagatgtggtcctgtacaacaatgctgacggcatgtacgaggtgtccttctattccaatgccgtggtctcctatgatggcagcatcttctggctgccgcctgccatctacaagagcgcatgcaagattgaagtaaagcacttcccatttgaccagcagaactgcaccatgaagttccgttcgtggacctacgaccgcacagagatcgacttggtgctgaagagtgaggtggccagcctggacgacttcacacctagtggtgagtgggacatcgtggcgctgccgggccggcgcaacgagaaccccgacgactctacgtacgtggacatcacgtatgacttcatcattcgccgcaagccgctcttctacaccatcaacctcatcatcccctgtgtgctcatcacctcgctagccatccttgtcttctacctgccatccgactgtggcgagaagatgacgttgtgcatctcagtgctgctggcgctcacggtcttcctgctgctcatctccaagatcatgcctcccacctccctcgacgtgccgctcgtcggcaagtacctcatgttcaccatggtgcttgtcaccttctccatcgtcaccagcgtgtgcgtgctcaacgtgcaccaccgctcgcccaccacgcacaccatggcgccctgggtgaaggtcgtcttcctggagaagctgcccgcgctgctcttcatgcagcagccacgccatcattgcgcccgtcagcgcctgcgcctgcggcgacgccagcgtgagcgcgagggcgctggagccctcttcttccgcgaagccccaggggccgactcctgcacgtgcttcgtcaaccgcgcgtcggtgcaggggttggccggggccttcggggctgagcctgcaccagtggcgggccccgggcgctcaggggagccgtgtggctgtggcctccgggaggcggtggacggcgtgcgcttcatcgcagaccacatgcggagcgaggacgatgaccagagcgtgagtgaggactggaagtacgtcgccatggtgatcgaccgcctcttcctctggatctttgtctttgtctgtgtctttggcaccatcggcatgttcctgcagcctctcttccagaactacaccaccaccaccttcctccactcagaccactcagcccccagctccaagtgaggcccttcctcatctccatgctctttcacccagccaccctctgctgcacagtagtgttgggtggaggatggacgagtgagctaccaggaagagggg

SEQ ID NO: 2 - protein, 502 aa, Homo sapiens
MARRCGPVALLLGFGLLRLCSGVWGTDTEERLVEHLLDPSRYNKLIRPATNGSELVTVQLMVSLAQLISVHEREQIMTTNVWLTQEWEDYRLTWKPEEFDNMKKVRLPSKHIWLPDVVLYNNADGMYEVSFYSNAVVSYDGSIFWLPPAIYKSACKIEVKHFPFDQQNCTMKFRSWTYDRTEIDLVLKSEVASLDDFTPSGEWDIVALPGRRNENPDDSTYVDITYDFIIRRKPLFYTINLIIPCVLITSLAILVFYLPSDCGEKMTLCISVLLALTVFLLLISKIMPPTSLDVPLVGKYLMFTMVLVTFSIVTSVCVLNVHHRSPTTHTMAPWVKVVFLEKLPALLFMQQPRHHCARQRLRLRRRQREREGAGALFFREAPGADSCTCFVNRASVQGLAGAFGAEPAPVAGPGRSGEPCGCGLREAVDGVRFIADHMRSEDDDQSVSEDWKYVAMVIDRLFLWIFVFVCVFGTIGMFLQPLFQNYTTTTFLHSDHSAPSSK

SEQ ID NO: 3 - DNA, 19 bp, Homo sapiens
ttcacacctagtggtagtg

SEQ ID NO: 4 - DNA, 20 bp, Homo sapiens
tggctgctgcatgaagagca

SEQ ID NO: 5 - DNA, 20 bp, Homo sapiens
ctgctgctcatctccaagat

(19) United States
(12) Patent Application Publication
(10) Pub. No.: US 2005/0032155 A1
(43) Pub. Date: Feb. 10, 2005

(54) Mutation in the beta2 nicotinic acetycholine receptor subunit associated with nocturnal frontal lobe epilepsy

(76) Inventors: Hilary Anne Philips, Port Norlunga (AU); John Charles Mulley, Firle (AU); Samuel Frank Berkovic, Caulfield (AU)

Correspondence Address: JENKINS & WILSON, PA, 3100 TOWER BLVD, SUITE 1400, DURHAM, NC, 27707, US

(21) Appl. No.: 10/275,858

(22) PCT Filed: May 11, 2001
(86) PCT No.: PCT/AU01/00541

(30) Foreign Application Priority Data
May 12, 2000 (AU) PQ 7491

(51) Int. Cl. C07K 14/705; C07H 21/04

(52) U.S. Cl. 435/69.1; 435/320.1; 435/325; 530/350; 536/23.5

(57) ABSTRACT

A point mutation in the β2 subunit of an nicotinic acetylcholine receptor providing a V287M transition is associated with autosomal dominant nocturnal frontal lobe epilepsy (ADNFLE).

### TECHNICAL FIELD

The present invention relates to mutations in the nicotinic acetylcholine receptor which are associated with idiopathic epilepsies in particular, with autosomal dominant nocturnal frontal lobe epilepsy (ADNFLE).

### BACKGROUND ART

Epilepsies constitute a diverse collection of brain disorders that affect about 3% of the population at some time in their lives (Annegers, 1996). An epileptic seizure can be defined as an episodic change in behaviour caused by the disordered firing of populations of neurons in the central nervous system. This results in varying degrees of involuntary muscle contraction and often a loss of consciousness. Epilepsy syndromes have been classified into more than 40 distinct types based upon characteristic symptoms, types of seizure, cause, age of onset and EEG patterns (Commission on Classification and Terminology of the International League Against Epilepsy, 1989). However the single feature that is common to all syndromes is the persistent increase in neuronal excitability that is both occasionally and unpredictably expressed as a seizure.

A genetic contribution to the aetiology of epilepsy has been estimated to be present in approximately 40% of affected individuals (Gardiner, 2000). As epileptic seizures may be the end-point of a number of molecular aberrations that ultimately disturb neuronal synchrony, the genetic basis for epilepsy is likely to be heterogeneous. There are over 200 Mendelian diseases which include epilepsy as part of the phenotype. In these diseases, seizures are symptomatic of underlying neurological involvement such as disturbances in brain structure or function. In contrast, there are also a number of “pure” epilepsy syndromes in which epilepsy is the sole manifestation in the affected individuals. These are termed idiopathic and account for over 60% of all epilepsy cases.

Idiopathic epilepsies have been further divided into partial and generalized sub-types. Partial (focal or local) epileptic fits arise from localized cortical discharges, so that only certain groups of muscles are involved and consciousness may be retained (Sutton, 1990). However, in generalized epilepsy, EEG discharge shows no focus such that all subcortical regions of the brain are involved. Although the observation that generalized epilepsies are frequently inherited is understandable, the mechanism by which genetic defects, presumably expressed constitutively in the brain, give rise to partial seizures is less clear. Certainly the study and isolation of the genes involved in rare families with primarily monogenic aetiology will aid in understanding the types of genes involved and the mechanisms of the disease process in general.

One such form of idiopathic partial epilepsy inherited in a simple Mendelian manner is called autosomal. dominant nocturnal frontal lobe epilepsy (ADNFLE). This epilepsy, first described in 1994 (Scheffer et al., 1994) is characterized by clusters of frontal lobe motor seizures occurring during sleep, with onset usually occurring in childhood. The condition is clinically distinctive and relatively homogeneous, although seizure severity and specific frontal lobe seizure manifestations vary within families (Hayman et al., 1997). Misdiagnosis as nightmares, night terrors, hysteria, sleep paralysis, other parasomnias, or even psychiatric disorders is common if clinicians are unaware of ADNFLE.

Linkage analysis of a large family with this form of epilepsy identified a locus on chromosome 20q13.2 most likely to contain the responsible gene (Phillips et al., 1995). Using a positional candidate approach, the neuronal nicotinic acetylcholine receptor α4 subunit (CHRNA4) was an ideal candidate gene due to the involvement of the nicotinic acetylcholine receptor (nAChR) in chemo-electrical transduction at cholinergic synapses of the central nervous system (CNS) and the fact that CHRNA4 is expressed in all layers of the frontal cortex (Wevers et al., 1994).

The nAChR is a transmembrane pentamer that is composed of up to four different subunits (α, β, γ, δ). The nAChRs are found not only in the nervous system but also in skeletal muscle, however in nerve cells, only two types of subunits, α and β, have been identified. Eleven distinct genes encoding neuronal nAChR subunits (α2-α9 and β2-β4) have been found in various species to date with the most abundant nAChR subtype in mammalian brain :consisting of two α4 and three β2 (CHRNB2) subunits (Schoepfer et al., 1988; Whiting et al., 1991; Sargent, 1993).

Each subunit of the nAChR consists of a long extracellular domain at the N terminus and four hydrophobic segments (M1-M4) that have sufficient length to traverse the membrane (reviewed by Jackson, 1999). These subunits associate together into a rosette-like structure with a water-filled pore in the middle. This membrane-spanning pore is lined by five α-helical segments constituting the M2 domains from each of the 5 subunits. These domains, in the absence of the neurotransmitter, ACh, appear to come together near the middle of the membrane and form the gate of the channel. The gate opens upon binding of ACh to distant sites on the α subunits allowing the flow of ions through the channel and closes again when ACh is depleted from the synaptic cleft or when desensitization of the receptor occurs. The M2 segment thus is a site where much of the action occurs during channel gating, indicating that this domain plays a pivotal role in the process of receptor activation.

Mutation analysis of the CHRNA4 gene in affected family members showing linkage to this locus identified a missense mutation that replaces serine with phenylalanine at codon 248, a strongly conserved amino acid in the second transmembrane domain (M2) of the encoded protein (Steinlein et al., 1995). This mutation was present in all 21 available affected members of the family as well as in 4 obligate carriers and the amino acid change was not seen in 333 healthy control subjects. A second mutation in this gene was identified in a Norwegian family with the same form of epilepsy, namely an insertion of three nucleotides at position 776, which encodes a leucine (Steinlein et al., 1997). This amino acid insertion again affected the M2 domain of the CHRNA4 protein. Physiological and pharmacological investigations of human nAChRs reconstituted in Xenopus oocytes with the control or mutated α4 subunits established that both mutations resulted in major but different changes to the receptors. The S248F mutation mainly affected the desensitization properties of the receptor while the leucine insertion increased the probability of transition to the active state (Bertrand et al., 1998). Although these mutations appeared to differentially affect the receptor properties they both result in reduced permeability to calcium and enhanced desensitization sensitivity that might account for the ADNFLE phenotype.

Genetic linkage studies of additional families with ADNFLE have suggested that they do not show linkage to the CHRNA4 locus at 20q13.2 (Berkovic et al., 1995; Phillips et al., 1998). While one ADNFLE family showed linkage to 15q24 (close to the CHRNA3/CHRNA5/CHRNB4 gene cluster), in other families both 15q24 and 20q13.2 were excluded indicating that at least three different genes exist accounting for ADNFLE. In fact, in at least three other forms of monogenic idiopathic epilepsy, locus heterogeneity has also been demonstrated. These include benign familial neonatal convulsions (BFNC) mapped to 20q13.2 and 8q(Leppert et al., 1989; Lewis et al., 1993), familial febrile seizures mapped to 8q13-q21and 19p13.3 (Wallace et al., 1996; Johnson et al., 1998) and benign familial infantile convulsions mapped to 19q and 16 (Guipponi et al., 1997; Szepetowski et al., 1997). For BFNC, the genes located in the 20q13.2 and 8q critical regions that were found to be mutated in individuals with the disease were homologous potassium channels (Biervert et al., 1998; Charlier et al., 1998; Singh et al., 1998).

### DISCLOSURE OF THE INVENTION

The present inventors have found that the CHRNB2 locus is involved in autosomal dominant nocturnal frontal lobe epilepsy (ADNFLE), and so implicated the β-subunits of the nAChR in idiopathic epilepsies.

According to one aspect of the present invention there is provided an isolated DNA molecule encoding a mutant β-subunit of a mammalian nicotinic acetylcholine receptor (nAchR), wherein a mutation event selected from the group consisting of point mutations, deletions, insertions and rearrangements has occurred in the nucleotides encoding the M2 domain of the β-subunit of said mammalian nicotinic acetylcholine receptor and said mutation event disrupts the functioning of an assembled mammalian nicotinic acetylcholine receptor comprising the β-subunit so as to produce an epilepsy phenotype.

Preferably said mutation event is a point mutation. The mutation typically results in replacement of valine residue. The valine residue is typically replaced by an amino acid having a more bulky side chain and/or a β-carbon atom substituted only by hydrogen atoms, of which methionine and leucine are preferred. The valine residue typically forms part of the lining of the ion channel in the vicinity of the opening of the channel to the synaptic cleft.

Advantageously said valine is V287 using nomenclature on the NCBI database (V262 in the numbering of Rempel et al (1998)), which occurs as a result of a G to A nucleotide transition at base 1025, as shown in SEQ ID NO:1. The G to A nucleotide transition creates a NlaIII restriction enzyme site.

The present invention also encompasses DNA molecules in which one or more additional mutation events selected from the group consisting of point mutations, deletions, insertions and rearrangements have occurred. Any such DNA molecule will have the mutation associated with epilepsy described above and will be functional, but otherwise may vary significantly from the DNA molecules set forth in SEQ ID NO:1.

The nucleotide sequences of the present invention can be engineered using methods accepted in the art for a variety of purposes. These include, but are not limited to, modification of the cloning, processing, and/or expression of the gene product. PCR reassembly of gene fragments and the use of synthetic oligonucleotides allow the engineering of the nucleotide sequences of the present invention. For example, oligonucleotide-mediated site-directed mutagenesis can introduce further mutations that create new restriction sites, alter expression patterns and produce splice variants etc.

As a result of the degeneracy of the genetic code, a number of polynucleotide sequences, some that may have minimal similarity to the polynucleotide sequences of any known and naturally occurring gene, may be produced. Thus, the invention includes each and every possible variation of a polynucleotide sequence that could be made by selecting combinations based on possible codon choices. These combinations are made in accordance with the standard triplet genetic code as applied to the polynucleotide sequences of the present invention, and all such variations are to be considered as being specifically disclosed.

The DNA molecules of this invention include cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences possessing a substantially different codon usage than that of the polynucleotide sequences of the present invention. For example, codons may be selected to increase the rate of expression of the peptide in a particular prokaryotic or eukaryotic host corresponding with the frequency that particular codons are utilized by the host. Other reasons to alter the nucleotide sequence without altering the encoded amino acid sequences include the production of RNA transcripts having more desirable properties, such as a greater half-life, than transcripts produced from the naturally occurring mutated sequence.

The invention also encompasses production of DNA sequences of the present invention entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding the polypeptides of the present invention. In cases where the complete coding sequence, including the initiation codon and upstream regulatory sequences, are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

The invention also includes nucleic acid molecules that are the complements of the sequences described herein.

According to another aspect of the present invention there is provided an isolated DNA molecule comprising the nucleotide sequence set forth in SEQ ID NO:1.

According to still another aspect of the present invention there is provided an isolated DNA molecule consisting of the nucleotide sequence set forth in SEQ ID NO:1.

The present invention allows for the preparation of purified polypeptide or protein from the polynucleotides of the present invention, or variants thereof. In order to do this, host cells may be transformed with a DNA molecule as described above. Typically said host cells are transfected with an expression vector comprising a DNA molecule according to the invention. A variety of expression vector/host systems may be utilized to contain and express sequences encoding polypeptides of the invention. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express a protein using a vaccinia virus expression system. The invention is not limited by the host cell employed.

The polynucleotide sequences, or variants thereof, of the present invention can be stably expressed in cell lines to allow long term production of recombinant proteins in mammalian systems. Sequences encoding the polypeptides of the present invention can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of the gene are needed, such as for antibody production, vectors which direct high levels of expression of this protein may be used, such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate cDNA sequence is inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence. The desired protein is then obtained by enzymatic cleavage of the fusion protein.

Fragments of the polypeptides of the present invention may also be produced by direct peptide synthesis using solid-phase techniques. Automated synthesis may be achieved by using the ABI 431A Peptide Synthesizer (Perkin-Elmer). Various fragments of this protein may be synthesized separately and then combined to produce the full length molecule.

According to still another aspect of the present invention there is provided an isolated polypeptide, said polypeptide being a mutant β-subunit of a mammalian nicotinic acetylcholine receptor (nAChR), wherein a mutation event selected from the group consisting of substitutions, deletions, insertions and rearrangements has occurred in the M2 domain and said mutation event disrupts the functioning of an assembled mammalian nicotinic acetylcholine receptor so as to produce an epilepsy phenotype.

Typically said mutation event is a substitution involving a valine residue, which is generally substituted by an amino acid having a more bulky side chain and/or β-carbon atom substituted only by hydrogen atoms. Typically said valine residue is replaced by methionine or leucine.

Preferably the substitution is a V287M transition as illustrated in SEQ ID NO:2.

The isolated polypeptide of the present invention may have been subjected to one or more mutation events selected from the group consisting of substitutions, deletions, insertions and rearrangements in addition to the mutation associated with epilepsy. Typically these mutation events are conservative substitutions.

According to still another aspect of the present invention there is provided an isolated polypeptide comprising the sequence set forth in SEQ ID NO:2.

According to still another aspect of the present invention there is provided a polypeptide consisting of the amino acid sequence set forth in SEQ ID NO:2.

According to still another aspect of the present invention there is provided an isolated polypeptide, said polypeptide being an assembled mammalian nicotinic acetylcholine receptor, comprising at least one α-subunit and at least one β-subunit, wherein a mutation event selected from the group consisting of substitutions, deletions, insertions and rearrangements has occurred in the M2 domain of a β-subunit and said mutation event disrupts the functioning of the assembled mammalian nicotinic acetylcholine receptor so as to produce an epilepsy phenotype.

The assembled nicotinic acetylcholine receptor may contain mutations in a single β-subunit or in a number of β-subunits. The main functional nAchR in the brain is a pentameric molecule consisting of two α4 subunits and three β2 subunits, and a mutation event may have occurred in any or all of the β2 subunits.

According to still another aspect of the present invention there is provided a method of preparing a polypeptide, said polypeptide being a mutant β-subunit of a mammalian nicotinic acetylcholine receptor, comprising the steps of:

(1) culturing host cells transfected with an expression vector comprising a DNA molecule as described above under conditions effective for polypeptide production; and

(2) harvesting the mutant β-subunit.

The mutant β-subunit may also be allowed to assemble with wild-type β-subunits and other subunits of the nicotinic acetylcholine receptor, whereby the assembled nAChR is harvested.

Substantially purified protein or fragments thereof can then be used in further biochemical analyses to establish secondary and tertiary structure for example by X-ray crystallography of crystals of the proteins or by NMR. Determination of structure allows for the rational design of pharmaceuticals to interact with the nAChR through a specific subunit protein, alter the overall nAChR protein charge configuration or charge interaction with other proteins, or to alter its function in the cell.

It will be appreciated that, having identified a mutation involved in epilepsy (ADNFLE) in these proteins, the nAChRs will be useful in further applications which include a variety of hybridisation and immunological assays to screen for and detect the presence of either a normal or mutated gene or gene product.

The invention enables therapeutic methods for the treatment of epilepsy, particularly ADNFLE, and also enables methods for the diagnosis of idiopathic epilepsies.

According to still another aspect of the invention there is provided a method of treating epilepsy, comprising administering a selective antagonist of the nicotinic acetylcholine receptor when it contains a mutation in the M2 domain of a β-subunit, said mutation being causative of epilepsy, to a subject in need of such treatment.

In still another aspect of the invention there is provided the use of a selective antagonist of the nAChR when it contains a mutation in the M2 domain of β-subunit, said mutation being causative of epilepsy, in the manufacture of a medicament for the treatment of epilepsy.

In one aspect, an antibody, which specifically binds to a mutant nAChR, may be used directly as an antagonist or indirectly as a targeting or delivery mechanism for bringing a pharmaceutical agent to cells or tissues that express the nAChR.

In a still further aspect of the invention there is provided an antibody which is immunologically reactive with a polypeptide as described above, but not with a wild-type nicotinic acetylcholine receptor or subunit thereof.

In particular, there is provided an antibody to an assembled nAChR containing a mutation causative of epilepsy in the M2 domain of a β-subunit. The antibody may be a monoclonal antibody or polyclonal antibody as would be understood by the person skilled in the art.

Alternatively, in some mutants, it may be possible to prevent the disorder by introducing another copy of the homologous subunit gene bearing a second mutation in that gene, or to alter the mutation, or to use another gene to block any negative effect.

In a further aspect of the invention there is provided a method of treating epilepsy, comprising administering an isolated DNA molecule which is the complement of any one of the DNA molecules described above and which encodes a mRNA that hybridizes with the mRNA encoding the mutant β-subunits of the nAChR, to a subject in need of such treatment.

In a still further aspect of the invention there is provided the use of an isolated DNA molecule which is the complement of a DNA molecule of the invention and which encodes a mRNA that hybridizes with the mRNA encoding the mutant β-subunits of the nAChR, in the manufacture of a medicament for the treatment of epilepsy.

Typically, a vector expressing the complement of the polynucleotide encoding the subunits constituting the nAChR may be administered to a subject to treat or prevent epilepsy, particularly ADNFLE. Antisense strategies may use a variety of approaches including the use of antisense oligonucleotides, injection of antisense RNA and transfection of antisense RNA expression vectors. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (For example, see Goldman et al., 1997).

In further embodiments, any of the antagonists, antibodies, complementary sequences or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of the various disorders described above. Using this approach, therapeutic efficacy with lower dosages of each agent may be possible, thus reducing the potential for adverse side effects.

Using methods well known in the art, a selective antagonist of a mutant nAChR may be produced. In particular, a mutant nAChR may be used to produce antibodies specific for the mutant β-subunits causative of the idiopathic epilepsies or to screen libraries of pharmaceutical agents to identify those that specifically bind the mutant nAChR. Such antibodies may include, but are not limited to, polyclonal, monoclonal, chimeric, and single chain antibodies.

For the production of antibodies, various hosts including rabbits, rats, goats, mice, humans, and others may be immunized by injection with a polypeptide as described or with any fragment or oligopeptide thereof (provided it includes the mutation of the invention) which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the mutant nAChR have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of nAChR amino acids may be fused with those of another protein, such as KLH, and antibodies to the chimeric molecule may be produced.

Monoclonal antibodies to a mutant nAChR may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for a nAChR may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a nAChR and its specific antibody. A two-site, monoclonal-based immunoassay utilizing monoclonal antibodies reactive to two non-interfering nAChR epitopes is preferred, but a competitive binding assay may also be employed.

According to still another aspect of the invention, peptides of the invention, particularly purified mutant nAChR polypeptide and cells expressing these, are useful for the screening of candidate pharmaceutical agents in a variety of techniques. It will be appreciated that therapeutic agents useful in the treatment of the idiopathic epilepsies such as ADNFLE are likely to show binding affinity to the polypeptides of the invention. Such techniques include, but are not limited to, high-throughput screening for compounds having suitable binding affinity to the mutant nAChR polypeptides (see PCT published application WO84/03564). In this stated technique, large numbers of small peptide test compounds can be synthesised on a solid substrate and can be assayed through nAChR polypeptide binding and washing. Bound nAChR polypeptide is then detected by methods well known in the art. In a variation of this technique, purified polypeptides of the invention can be coated directly onto plates to identify interacting test compounds. The invention also contemplates the use of competition drug screening assays in which neutralizing antibodies capable of specifically binding the mutant nAChR compete with a test compound for binding thereto. In this manner, the antibodies can be used to detect the presence of any peptide that shares one or more antigenic determinants of the mutant nAChR.

The invention is particularly useful for screening compounds by using the polypeptides of the invention in transformed cells, transfected oocytes or transgenic animals. A particular drug is added to the cells in culture or administered to a transgenic animal containing mutant nAChRs and the effect on the current of the receptor is compared to the current of a cell or animal containing the wild-type nAChR. Drug candidates that alter the current to a more normal level are useful for treating or preventing diseases associated with nAChRs.

Any of the therapeutic methods described above may be applied to any subject in need of such therapy, including, for example, mammals such as dogs, cats, cows, horses, rabbits, monkeys, and most preferably, humans.

Polynucleotide sequences encoding a nAChR may be used for the diagnosis of the idiopathic epilepsies such as ADNFLE and the use of the DNA molecules of the invention in diagnosis of epilepsy, or a predisposition to epilepsy, is therefore contemplated.

In another embodiment of the invention, the polynucleotides that may be used for diagnostic purposes include oligonucleotide sequences, genomic DNA and complementary RNA and DNA molecules. The polynucleotides may be used to detect and quantitate gene expression in biological samples. Genomic DNA used for the diagnosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, hybridization using specific oligonucleotides, PCR mapping, RNase protection, and various other methods may be employed. For instance restriction enzyme digest and mapping can be employed for the specific G to A mutation in the CHRNB2 subunit described in this invention. The G to A transition in the M2 domain of this subunit creates a NlaIII restriction site. The DNA from an affected individual as well as a normal control may be amplified using oligonucleotides described in SEQ ID NO: 3 and 4. The amplification product may then be digested by NlaIII to provide a fingerprint for comparison to the DNA fingerprint of wild-type CHRNB2. In addition, direct nucleotide sequencing of amplification products from the nAChR can be employed. Sequence of the sample amplicon is compared to that of the wild-type amplicon to determine the presence (or absence) of nucleotide differences.

According to a further aspect of the invention there is provided the use of a polypeptide as described above in the diagnosis of epilepsy.

When a diagnostic assay is to be based upon proteins constituting a nAChR, a variety of approaches are possible. For example, diagnosis can be achieved by monitoring differences in the electrophoretic mobility of normal and mutant proteins that form the nAChR. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind mutant nAChRs may be used for the diagnosis of epilepsy, or in assays to monitor patients being treated with a complete nAChR or agonists, antagonists, or inhibitors of a nAChR. Antibodies useful for diagnostic purposes may be prepared in the same manner as described above for therapeutics. Diagnostic assays for nAChRs include methods that utilize the antibody and a label to detect a mutant nAChR in human body fluids or in extracts of cells or tissues. The antibodies may be used with or without modification, and may be labelled by covalent or non-covalent attachment of a reporter molecule.

A variety of protocols for measuring the presence of mutant nAChRs, including ELISAs, RIAs, and FACS, are known in the art and provide a basis for diagnosing epilepsies such as ADNFLE. The expression of a mutant receptor is established by combining body fluids or cell extracts taken from test mammalian subjects, preferably human, with antibody to the receptor under conditions suitable for complex formation. The amount of complex formation may be quantitated by various methods, preferably by photometric means. Antibodies specific for the mutant receptor will only bind to individuals expressing the said mutant receptor and not to individuals expressing only wild-type receptors (ie normal individuals). This establishes the basis for diagnosing the disease.

Once an individual has been diagnosed with epilepsy, effective treatments can be initiated. These may include administering a selective antagonist to the mutant receptor such as an antibody or mutant complement as described above. This therapy can also be supported with the introduction of wild-type receptor, particularly through gene therapy approaches. Typically, a vector capable of a expressing the appropriate full length nAChR subunit or a fragment of derivative thereof may be administered. The expression vector must be able to drive its own expression such that the level of normal protein will be sufficient for normal receptor formation.

In an alternative support approach to therapy, a substantially purified nAChR or nAChR subunit polypeptide and a pharmaceutically acceptable carrier may be administered. Pharmaceutical compositions in accordance with the present invention are prepared by mixing nAChR or nAChR subunit polypeptide or active fragments or variants thereof having the desired degree of purity, with acceptable carriers, excipients, or stabilizers which are well known. Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

Any of the proteins, antagonists, antibodies, agonists, complementary sequences, or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of epilepsy. Using this approach, therapeutic efficacy with lower dosages of each agent may be possible, thus reducing the potential for adverse side effects.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (For example, see Schena et al., 1996; Heller et al., 1997).

The present invention also provides for the production of genetically modified (knock-out or knock-in), non-human animal models transformed with the DNA molecules of the invention. These animals are useful for the study of the function of a nAChR, to study the mechanisms of disease as related to a nAChR, for the screening of candidate pharmaceutical compounds, for the creation of explanted mammalian cell cultures which express the mutant nAChR and for the evaluation of potential therapeutic interventions.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to their relative ease of maintenance and shorter life spans. For certain studies, transgenic yeast or invertebrates may be suitable and preferred because they allow for rapid screening and provide for much easier handling. For longer term studies, non-human primates may be desired due to their similarity with humans.

To create an animal model for a mutated nAChR several methods can be employed. These include generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create a transgenic mouse, which is preferred, a mutant version of a particular nAChR subunit can be inserted into a mouse germ line using standard techniques of oocyte microinjection or transfection or microinjection into embryonic stem cells. Alternatively, if it is desired to inactivate or replace an endogenous nAChR subunit gene, homologous recombination using embryonic stem cells may be applied.

For oocyte injection, one or more copies of the mutant nAChR subunit gene can be inserted into the pronucleus of a just-fertilized mouse oocyte. This oocyte is then reimplanted into a pseudo-pregnant foster mother. The liveborn mice can then be screened for integrants using analysis of tail DNA for the presence of the particular human subunit gene sequence. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

According to still another aspect of the invention there is provided the use of genetically modified non-human animals for the screening of candidate pharmaceutical compounds.

It will be clearly understood that, although a number of prior art publications are referred to herein, this reference does not constitute an admission that any of these documents forms part of the common general knowledge in the art, in Australia or in any other country.

Throughout this specification and the claims, the words “comprise”, “comprises” and “comprising” are used in a non-exclusive sense, except where the context requires otherwise.

### BRIEF DESCRIPTION OF THE DRAWINGS

Preferred forms of the invention are described, by way of example only, with reference to the following examples and the accompanying drawings, in which:

FIG. 1 is a chart of the lineage of a Scottish family showing the family members with the mutated CHRNB2 amino acid 287 (indicated by m);

FIG. 2 is a trace of the DNA sequence of CHRNB2 showing the c1025G-A transition in which the upper chromatogram shows the mutation and the lower chromatogram shows the control sequence;

FIG. 3 shows the alignment of various genes in order to allow a comparison of the homologies at the M2 CHRNB2 domain in which amino acid 287 is indicated by the box; and

FIG. 4 shows A, left panel, Neuronal α4β2 heteropentameric receptor resulting from the assembly of two α4 and three β2 subunits. Right panel, α4β2 is shown as a pentamer with two potential ACh-blinding sites. “β2*” identifies the V287M amino acid substitution. The ACh receptor can assemble without any mutated β2 subunit (α4β2 wild-type receptor) or with one, two, or three mutated β2* subunits. B, upper panel, Current traces elicited by consecutive applications of four or five increasing ACh concentrations (horizontal bars). The values above each bar indicate the concentration of ACh applied to the cell. Lower panel, Differences in ACh affinity are emphasized in a log-log plot. The logarithm of the absolute value of the current (measured in the upper panel, Log (−I) is plotted as a function of the logarithm of ACh concentrations (Log [ACh]). −I and [ACh] are expressed in nA and nM, respectively. Values are mean ±SEM, with n=9 (α4β2), n=17 (α4β2*), and n=10 (α4[β2*+β2]). Solid lines are the best linear regression throughout data points. C, upper panel, Representative macroscopic currents recorded in oocytes expressing either the wild-type or mutated β2 subunit. Currents evoked by four ACh concentrations (0.1, 0.2, 0.8 and 8 μM) are superimposed. Cells were held at −100 mV and challenged with ACh (10 s) once every 120 s. Bars indicate ACh applications. Lower panel, ACh activation curves for α4β2, α4β2* and α4(β2*+β2]). Dose-response curves were normalized to the maximal current amplitude of each cell. Values are mean ±SEM. For each ACh concentration, 3-8 independent measurements were averaged.

### MODES FOR PERFORMING THE INVENTION

### EXAMPLE 1

### Clinical Diagnosis of Affected Family Members

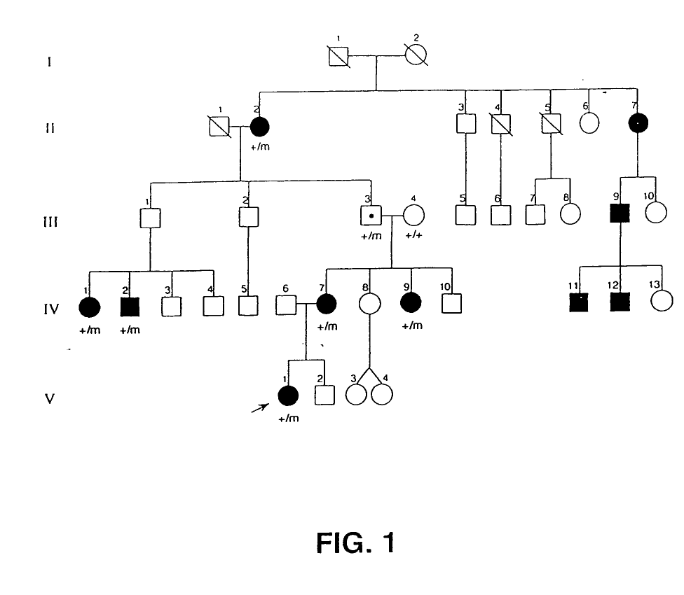

The proband (V-1; FIG. 1), a Caucasian female of normal intellect, presented at the age of 11 with nocturnal seizures. These would be typified by her waking from sleep with a sensation of difficulty in breathing. This would last a few seconds only, following which she would appear to be holding her breath and would make grunting noises. Sometimes she would recover quickly which would be followed by crying or screaming. On other occasions this would progress to tonic extension of her left arm and curling up into a ball. This would last up to a few minutes and the whole episode would be repeated stereotypically at around 15 minute intervals throughout the night. She would have a clear recollection of all of the above events. Such events would also occur during daytime sleep.

Video EEG telemetry showed no change during the ictus, although the EEG trace was largely obscured by muscle artifact. Inter-ictal recordings have on occasion shown some sharp disturbance in the right central parietal area.

Whilst her seizures were initially well controlled using carbamazepine, this relapsed and further control was only achieved with some difficulty. Currently, she is seizure free on a combination of phenytoin and topiramate.

In this family there are nine other affected members spanning four generations. The symptoms in these individuals have either been so mild that medical attention has not been sought or their symptoms have been easily controlled on carbamazepine. In some family members the seizures have remitted spontaneously but the oldest surviving family member (II-2; FIG. 1), at the age of 81, continues to have occasional seizures whilst on phenytoin.

### EXAMPLE 2

### Mutation Analysis of CHRNB2

The second transmembrane domains of the CHRNB2 gene were screened by direct sequencing of DNA obtained with consent from eight members of the family: six affected individuals, one obligate carrier and his unaffected wife (FIG. 1). DNA was extracted from peripheral blood samples using a method adapted from Wyman and White (1980). CHRNB2 specific primers used to amplify the extracted DNA are listed as SEQ ID NO: 3 and 4. These primers amplify a 468 base pair segment of the CHRNB2 gene that incorporates the M2 domain. PCR reactions contained 67 mM Tris-HCl (pH 8.8); 16.5 mM (NH₄)₂SO₄; 6.5 μM EDTA; 1.5 mM MgCl₂; 200 μM each DNTP; 10% DMSO; 0.17 mg/ml BSA; 10 mM β-mercaptoethanol; 15 μg/ml each primer; 100 U/ml Taq DNA polymerase, and 10 μg/ml genomic DNA. PCR reactions were performed using 10 cycles of 94° C. for 60 seconds, 60° C. for 90 seconds, and 72° C. for 90 seconds followed by 25 cycles of 94° C. for 60 seconds, 55° C. for 90 seconds, and 72° C. for 90 seconds. A final extension reaction for 10 minutes at 72° C. followed.

PCR amplified templates were purified for sequencing using QiaQuick PCR preps (Qiagen) following manufacturers procedures. The primers used to sequence the purified CHRNB2 PCR fragments were identical to those used for the initial amplification step (SEQ ID Numbers: 3 and 4). For each sequencing reaction, 25 ng of primer and 100 ng of purified PCR template were used. The BigDye sequencing kit (ABI) was used for all sequencing reactions according to the manufacturers specifications. The products were run on an ABI 377 Sequencer and analysed using the EditView program.

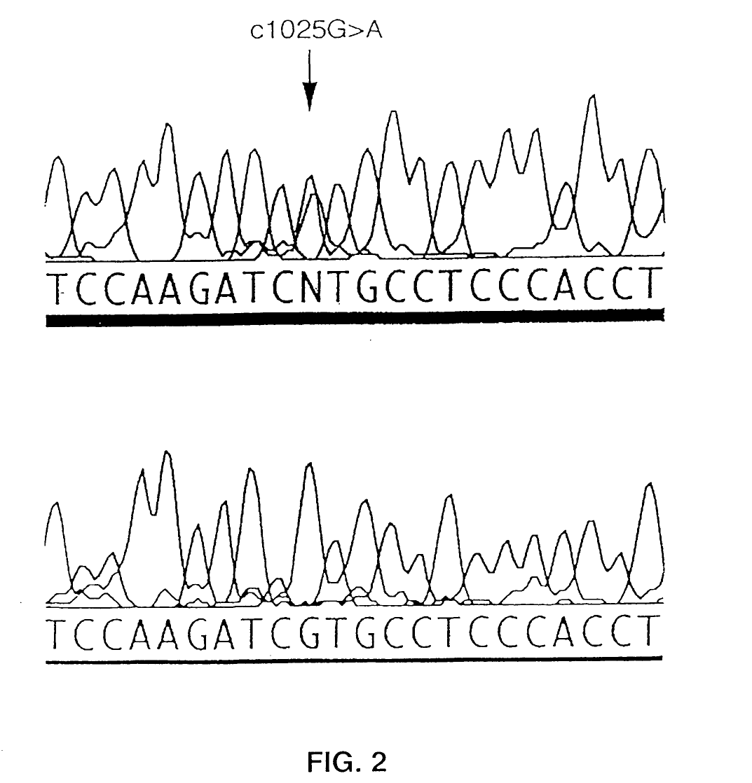

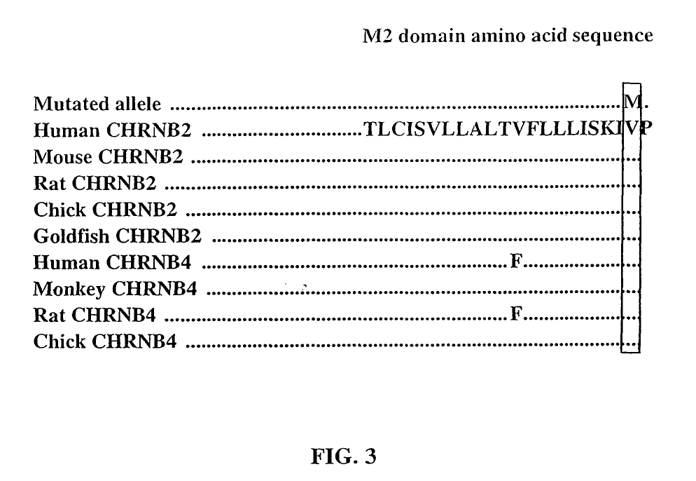

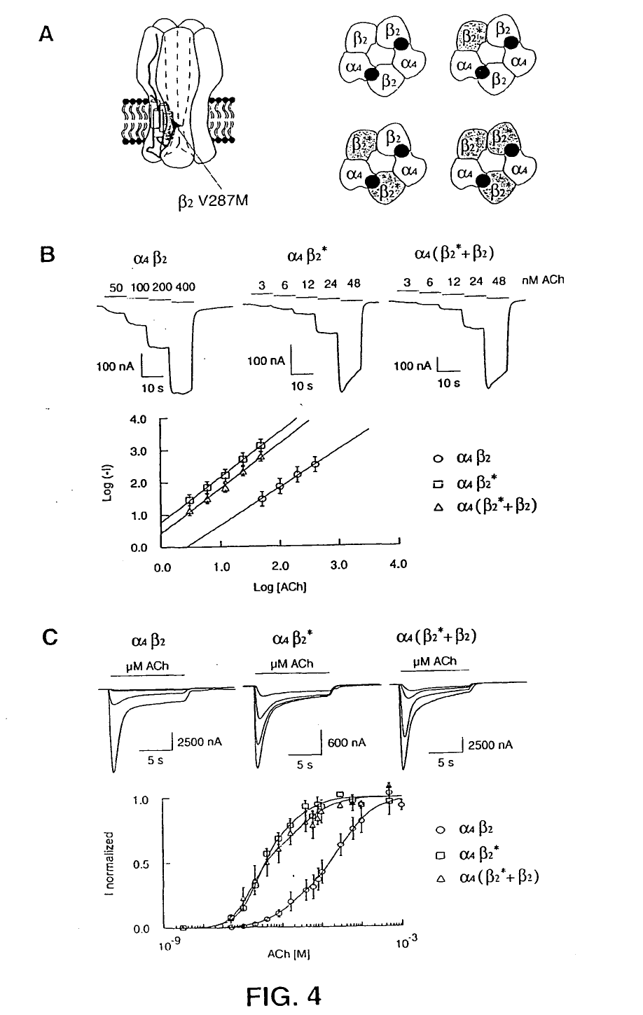

The sequencing strategy revealed a G→A transition in the M2 domain of CHRNB2 in the proband and in other family members where the presence of the mutation has been indicated (FIG. 1). The nucleotide sequence of the mutated form of the CHRNB2 gene is represented by SEQ ID NO: 1. The c1025G→A mutation (FIG. 2) replaces a highly conserved valine with a methionine at position 287, using nomenclature on the NCBI database, or at position 262, using the numbering of Rempel et al., (1998). The amino acid sequence of the mutated form of the CHRNB2 gene is represented by SEQ ID NO: 2. All affected individuals and the unaffected obligate carrier had the mutation There is much interspecies and between subunit homology among CHRN subunits, especially in the four transmembrane domains. The CHRNB subunits CHRNB2, CHRNB3 and CHRNB4 are all expressed in the brain. The M2 domain of CHRNB3 has only 59% homology with the other two and in vitro studies in the rat show that, when coexpressed with CHRNA subunits alone, CNRNB3 does not assemble into a functional receptor. The β-type subunits therefore may function in ion channels gated by ligands other than nicotine and acetylcholine (Willoughby et al., 1993). The M2 domains of CHRNB2 and CHRNB4 have almost complete homology and in particular valine287 is fully conserved in these subunits as well as in a number of other species (FIG. 3). CHRNB1, which is expressed only in muscle, has leucine at this position. This may indicate valine 287 is essential for normal ion channel function in the brain. The V287M mutation is located near the extracellular end of the M2 domain that lines the ionic pore (FIG. 4A). Valine 287 faces into the pore of the ion channel in the open and closed state (Devilliers-Thiery et al., 1993) and when valine 287 is replaced by a methionine there is an apparent 10-fold increase in Ach affinity.

### EXAMPLE 3

### Confirmation of the V287M Mutation—Restriction Enzyme Analysis

The G→A transition creates a NlaIII restriction site. Primers represented by SEQ ID Numbers: 3 and 4 amplify a 468 base pair fragment that contains four NlaIII sites present in normal alleles. Digestion of this normal amplicon with NlaIII will produce fragments of 318, 78, 54, 9 and 9 base pairs. However, digestion of an amplicon containing the G→A transition with NlaIII will produce fragments of 273, 78, 54, 45, 9 and 9 base pairs. The bands of 318 and 273 base pairs are easily detected on 2% agarose gels and therefore this system provides a mechanism to confirm the presence of the mutation in affected family members.

### EXAMPLE 4

### Confirmation of the V287M Mutation—SSCP Analysis

To confirm that the observed amino acid substitution found in the CHRNB2 M2 domain was specific to affected members of the studied family, single strand conformation polymorphism analysis was performed on DNA collected from 102 anonymous Australian blood donors. The primers used for PCR amplification of DNA from both control donor samples and affected family members are represented by SEQ ID Numbers: 4 and 5. These primers amplified a product of 220 base pairs using the following conditions: PCR reactions contained 67 mM Tris-HCl (pH 8.8); 16.5 mM (NH₄)₂SO₄; 6.5 μM EDTA; 1.5 mM MgCl₂; 200 μA each dNTP; 10% DMSO; 0.17 mg/ml BSA; 10 mM β-mercaptoethanol; 15 μg/ml each primer; 200 μCi/ml [α-32P]dCTP; 100 U/ml Taq DNA polymerase, and 10 μg/ml genomic DNA. PCR reactions were performed using 10 cycles of 94° C. for 60 seconds, 60° C. for 90 seconds, and 72° C. for 90 seconds followed by 25 cycles of 94° C. for 60 seconds, 55° C. for 90 seconds, and 72° C. for 90 seconds. A final extension reaction for 10 minutes at 72° C. followed. Completed PCR reactions were subsequently mixed with an equal volume of formamide loading buffer (96% formamide; 1 mM EDTA; 0.1% bromophenol blue; 0.1% xylene cyanol) and were heated to 95° C. for 3 minutes before snap cooling on ice. Five μl of each sample was then loaded onto gels containing 10% (49:1) polyacrylamide, 5% glycerol and TBE. The gels were run at 700 volts for 20 hours at room temperature, dried and exposed to X-ray film.

A bandshift was identified, however the shift was only observed in family members with the G→A transition and not in any of the 102 control DNA samples. The bandshift detected by SSCP analysis could only be associated with the G→A transition, since no other base changes were detected in the PCR product. The fact that the nucleotide change only occurs in affected family members and not in healthy controls suggests the change has a functional significance.

### EXAMPLE 5

### Functional Significance of the V287M Mutation

To define the effects of the β2 V287M mutation on the physiological properties of the α4β2 nAChR, the V287M amino acid substitution was introduced into the β2 coding sequence according to the PCR based strategy described by Nelson and Long (1989). The 411 base pair NheI/PmlI mutagenesis cassette was sequenced to verify the presence of the V287M mutation. Xenopus laevis oocytes at stage V or VI were isolated and nuclear-injected with 2 ng of DNA solution based on a standard procedure (Bertrand et al., 1991). For functional reconstitution of α4β2, α4β2V287M and α4(β2+β2V287M) receptors, cDNAs coding for CHRNA4 (Monteggia et al., 1995), CHRNB2 and CHRNB2-V287M subunits were mixed at molecular ratios of 1:1, 1:1 and 2:1:1 respectively. Following DNA injection, oocytes were maintained at 18° C. for 2 to 3 days in standard Barth's solution containing 88 mM NaCl, 1 mM KCl, 2.4 mM NaHCO₃, 0.8 mM MgSO₄, 0.3 mM Ca(NO₃)₂, 0.4 mM CaCl₂, 10 mM HEPES-NaOH, pH 7.4 and were complemented with kanamycin (20 μg/ml), penicillin (100 μg/ml) and streptomycin (100 μg/ml).

Macroscopic currents were recorded by a two-electrode voltage clamp at 18° C. using a GENECLAMP 500 amplifier (Axon Instruments). The borosilicate electrodes were filled with 3 M KCl and the oocytes were continuously perfused a solution containing 82.5 mM NaCl, 2.5 mM KCl, 2.5 mM CaCl₂, 1 mM MgCl₂, 0.5 μM atropine sulphate, 5 mM HEPES-NaOH, pH 7.4. Ach (Fluka) was diluted in this solution for the subsequent experiments. Gravity driven solutions were applied to the recording chamber through computer controlled electromagnetic valves. For all experiments, the holding potential was −100 mV.

When challenged with saturating concentrations of Ach, the α4β2 and α4β2V287M containing receptors yielded robust currents (>5 μA). However, at low concentrations of Ach, distinction between these two types of receptors was observed. With oocytes containing the α4β2V287M subunits, 3 nM Ach was already sufficient to activate reliable ionic currents (81±24 nA, n=18) compared with wild-type receptors which needed 50 nM Ach to evoke currents of similar amplitude (65±22 nA, n=9)(FIG. 4B).

To characterize these differences further, currents evoked by four or five consecutive applications of increasing non- or slightly-desensitizing Ach concentrations was recorded. Representative current recordings are shown in the upper panel of FIG. 4B. The lower panel of FIG. 4B shows a plot of the logarithm of Ach-evoked current (absolute value) versus the logarithm of agonist concentrations which indicated the expected linear relationship. The α4β2V287M exhibited ˜1 order of magnitude higher Ach sensitivity than did the wild-type receptors which confirmed the initial qualitative observation that mutated receptors are more sensitive to Ach.

To further examine Ach sensitivity, the dose-response relationship over a broad range of concentrations was determined for both control and mutated receptors. FIG. 4C (upper panel) shows superimposed currents evoked by 0.1, 0.2, 0.8 and 8 μM external Ach. The Ach sensitivity was determined by plotting peak current versus the logarithm of agonist concentration (FIG. 4C, lower panel). Observation of the α4β2 activation curves suggest that they are best described by the sum of two Hill equations (See below, solid lines in FIG. 4C, lower panel). Most of the Ach activation curves appeared biphasic with high- and low-affinity components and consequently Ach dose-response relationships are best fitted to the sum of two empirical Hill equations:
I=Iₘₐₓ{[a/(1+EC50H/[ACh])^{nH}]+(1−a)/(1+EC50L/[ACh]^{nL})}
where I_{maw} is the amplitude of the maximal current elicited by ACh application, EC50 is the ACh concentration for half-maximal current activation, [ACh] is the concentration of ACh, and n is the Hill coefficient. Parameters relating to the high- and low-affinity component are identified by H and L respectively. Parameter a is the relative contribution of the high-affinity component to the total current response over the range of concentrations and is expressed as the fraction of the high-affinity sites. Table 1 shows the values for these parameters. Within the limits of the model used for the data fit, the α4β2V287M receptor exhibited both a decrease in the EC50s and an increase in the relative contribution of the high-affinity component. Thus, both log-log plot and dose-response curves accounted for a higher apparent ACh affinity associated with the β2V287M mutation.

The peak versus plateau current ratio revealed no major differences between the wild-type and mutated receptors indicating that the V287M mutation causes no significant alteration of desensitization properties.

As the affected family members of the present study are heterozygous for the β2 gene, all cells from these individuals most likely express both wild-type and mutated subunits. Therefore experiments that examine the effect of co-injection of both wild-type and mutated β2 subunits in the same oocyte are essential. Results showed that within the same batch of oocytes, currents evoked by saturating ACh concentrations were indistinguishable on the basis of their amplitude. In contrast, an obvious difference was observed for the apparent ACh affinity. Both the high and low EC50 values were comparable to receptors containing three V287M β2 subunits (Table 1). In addition, α4(β2+β2V287M), like α4β2V287M exhibited a higher affinity in the log-log plot (FIG. 4B).

These findings are consistent with the autosomal dominant mode of inheritance of ADNFLE. FIG. 4A indicates that four distinct subunit combinations are possible when both wild-type and mutated subunits are expressed in a cell. Based on this, 75% of the receptors will contain a mutated β2 subunit and the dominant effect of the mutation can therefore be easily accounted for. As oocytes that express heterozygote or homozygote combinations of the mutated β2 subunit display very similar properties, the presence of a single β2 mutation within a receptor complex may be sufficient to confer the properties associated with the V287M substitution.

### EXAMPLE 6

### Analysis of the nAChR and Receptor Subunits

The following methods are used to determine the structure and function of the nAChR and receptor subunits.

Molecular Biological Studies

The ability of nAChR as a whole or through individual subunits to bind known and unknown protein can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

The nature of the nAChR interacting genes and proteins can also be studied such that these partners can also be targets for drug discovery.

Structural Studies

nAChR recombinant proteins can be produced in bacterial, yeast, insect and/or mammalian cells and used in crystallographical and NMR studies. Together with molecular modelling of the protein, structure-driven drug design can be facilitated.

### EXAMPLE 7

### Generation of Polyclonal Antibodies Against nAChR Subunits

Following the identification of a mutation in the β2 subunit of the nAChR in ADNFLE and therefore confirming the involvement of the receptor in epilepsy, antibodies can be made to selectively bind and distinguish mutant from normal protein. Antibodies specific for mutagenised epitopes are especially useful in cell culture assays to screen for cells which have been treated with pharmaceutical agents to evaluate the therapeutic potential of the agent.

To prepare polyclonal antibodies, short peptides can be designed homologous to a particular nAChR subunit amino acid sequence. Such peptides are typically 10 to 15 amino acids in length. These peptides should be designed in regions of least homology to the mouse orthologue to avoid cross species interactions in further down-stream experiments such as monoclonal antibody production. Synthetic peptides can then be conjugated to biotin (Sulfo-NHS-LC Biotin) using standard protocols supplied with commercially available kits such as the PIERCE™ kit (PIERCE). Biotinylated peptides are subsequently complexed with avidin in solution and for each peptide complex, 2 rabbits are immunized with 4 doses of antigen (200 μg per dose) in intervals of three weeks between doses. The initial dose is mixed with Freund's Complete adjuvant while subsequent doses are combined with Freund's Immuno-adjuvant. After completion of the immunization, rabbits are test bled and reactivity of sera assayed by dot blot with serial dilutions of the original peptides. If rabbits show significant reactivity compared with pre-immune sera, they are then sacrificed and the blood collected such that immune sera can separated for further experiments.

This procedure is repeated to generate antibodies against wild-type forms of receptor subunits. These antibodies, in conjunction with antibodies to mutant nAChR subunits, are used to detect the presence and the relative level of the mutant forms in various tissues.

### EXAMPLE 8

### Generation of Monoclonal Antibodies Specific for nAChR Subunits

Monoclonal antibodies can be prepared for nAChR subunits in the following manner. Immunogen comprising an intact nAChR subunit protein or nAChR subunit peptides (wild type or mutant) is injected in Freund's adjuvant into mice with each mouse receiving four injections of 10 to 100 ug of immunogen. After the fourth injection blood samples taken from the mice are examined for the presence of antibody to the immunogen. Immune mice are sacrificed, their spleens removed and single cell suspensions are prepared (Harlow and Lane, 1988). The spleen cells serve as a source of lymphocytes, which are then fused with a permanently growing myeloma partner cell (Kohler and Milstein, 1975). Cells are plated at a density of 2×10⁵ cells/well in 96 well plates and individual wells are examined for growth. These wells are then tested for the presence of nAChR subunit specific antibodies by ELISA or RIA using wild type or mutant subunit target protein. Cells in positive wells are expanded and subcloned to establish and confirm monoclonality. Clones with the desired specificity are expanded and grown as ascites in mice followed by purification using affinity chromatography using Protein A Sepharose, ion-exchange chromatography or variations and combinations of these techniques.

Industrial Applicability

The present invention allows for the diagnosis and treatment of idiopathic epilepsies such as ADNFLE.

TABLE 1
Properties of Wild-type and β2-V287M Containing Receptors

| Subunit | | | | | | | |
|---|---|---|---|---|---|---|---|
| Combinations | EC50 (H) | EC50 (L) | n (H) | n (L) | % (H) | % (L) | Number |
| α4β2 | 3.9 ± 1.3 | 47.6 ± 1.88 | 1.2 ± 0.2 | 1.3 ± 0.2 | 26 ± 5 | 74 ± 5 | 8 |
| α4β2* | 0.25 ± 0.04 | 2.9 ± 0.9 | 1.6 ± 0.1 | 1.9 ± 0.1 | 77 ± 1.5 | 23 ± 1.5 | 7 |
| α4 (β2* + β2) | 0.42 ± 0.14 | 5.3 ± 2.3 | 1.6 ± 0.1 | 1.0 ± 0.1 | 65 ± 3 | 35 ± 3 | 7 |

Note:
Sensitivity to ACh was measured in several cells (n >= 5). Values correspond to the best fits obtained with Equation 1.
Values are mean ± SEM.
*Subunits with the V287M mutation.
Equation 1:
I = Iₘₐₓ{[a/(1 + EC50H/[ACh])^{nH}] + (1 − a)/(1 + EC50L/[ACh]^{nL})}

References

References cited herein are listed on the following pages, and are incorporated herein by this reference.
Annegers, J F. (1996). The treatment of epilepsy: Principles and practice. Second Edition. (Wyllie E (Ed) Williams and Wilkins).
Aubert, I. et al. (1992). J. Neurochem. 58: 529-541.
Berkovic, S F. et al. (1995). Epilepsia 36, Suppl 4: 147.
Bertrand, D. et al. (1991). Electrophysiology of neuronal nicotinic acetylcholine receptors expressed in Xenopus oocytes following nuclear injection of genes or cDNAs. (Academic Press, New York).
Bertrand, S. et al. (1998). British J. Pharmacol. 125: 751-760.
Biervert, C. et al. (1998). Science 279: 403-406.
Charlier, C. et al. (1998). Nat. Genet. 18: 53-55.
Cole, S P. et al. (1984). Mol. Cell Biol. 62: 109-120.
Commission on Classification and Terminology of the International League Against Epilepsy. (1989). Epilepsia. 30: 389-399.
Cote, R J. et al. (1983). Proc. Natl. Acad. Sci. USA 80: 2026-2030.
Devilliers-Thiery, A. et al. (1993). J. Membr. Biol. 136: 97-112.
Gardiner, M. (2000). J Neurol. 247: 327-334.
Goldman, C K. et al. (1997). Nature Biotechnology 15: 462-466.
Guipponi, M. et al. (1997). Hum. Mol. Genet. 6: 473-477.
Harlow, E. and Lane, D. (1988). Antibodies: A Laboratory Manual (Cold Spring Harbor Laboratory, Cold Spring Harbor, New York).
Hayman, M. et al. (1997). Neurology 49: 969-975.
Heller, R A. et al. (1997). Proc. Natl. Acad. Sci. USA 94: 2150-2155.
Huse, W D. et al. (1989). Science 246: 1275-1281.
Jackson, M B. (1999). Jasper's Basic Mechanisms of the Epilepsies, Third Edition: Advances in Neurology 79: 511-524.
Johnson, E W. et al. (1998). Hum. Mol. Genet. 7: 63-68.
Kohler, G. and Milstein, C. (1975). Nature 256: 495-497.
Kozbor, D. et al. (1985). J. Immunol. Methods 81:31-42.
Leppert, M. et al. (1989). Nature 337: 647-648.
Lewis, T B. et al. (1993). Am. J. Hum. Genet. 53: 670-675.
Monteggia, L M. et al. (1995). Gene 155: 189-193.
Nelson, R M. and Long, GL. (1989). Anal. Biochem. 180: 147-151.
Orlandi, R. et al. (1989). Proc. Natl. Acad. Sci. USA 86: 3833-3837.
Phillips, H A. et al. (1995). Nature Genet. 10: 117-118.
Phillips, H A. et al. (1998). Am. J. Hum. Genet. 63: 1108-1116.
Rempel, N. et al. (1998). Hum. Genet. 103: 645-653.
Sambrook, J. et al. (1989). Molecular cloning: a laboratory manual. Second Edition. (Cold Spring Harbour Laboratory Press, New York).
Sargent, P B. (1993). Annu. Rev. Neurosci. 16: 403-443.
Scheffer, I E. et al. (1994). Pediat. Neurol. 11: 95.
Schena, M. et al. (1996). Proc. Natl. Acad. Sci. USA 93: 10614-10619.
Schoepfer, R. et al. (1988). Neuron 1: 241-248.
Schroder, H. et al. (1995). Alzheimer disease: therapeutic strategies. Giacobini E, Becker R E (Editors). (Birkhauser, Boston). pp181-185.
Singh, N A. et al. (1998). Nat. Genet. 18: 25-29.
Steinlein, O K. et al. (1995). Nature Genet. 11: 201-203.
Steinlein, O K. et al. (1997). Hum. Mol. Genet. 6: 943-948.
Sugaya, K. et al. (1990). J. Neurosci. Res. 27: 349-359.
Sutton, G C. (1990). The principles and practice of medical genetics. Second Edition. (Churchill Livingstone, N.Y.).
Szepetowski, P. et al. (1997). Am. J. Hum. Genet. 61: 889-898.
Wallace, R. et al. (1996). J. Med. Genet. 33: 308-312.
Wevers, A. et al. (1994). Brain Res. Molec. Brain Res. 25: 122-128.
Whiting, P J. et al. (1991). Mol. Brain Res. 10: 61-70.
Willoughby, J J. et al. (1993). Neurosci. Lett. 155: 136-139.
Winter, G. et al. (1991). Nature 349: 293-299.
Wyman, A R and White, R. (1980). Proc. Natl. Acad. Sci. 77: 6754-6758.

1. An isolated DNA molecule encoding a mutant β-subunit of a mammalian nicotinic acetylcholine receptor (nAchR), wherein a mutation event selected from the group consisting of point mutations, deletions, insertions and rearrangements has occurred in the nucleotides encoding the M2 domain of the β-subunit of said mammalian nicotinic acetylcholine receptor and said mutation event disrupts the functioning of an assembled mammalian nicotinic acetylcholine receptor comprising the β-subunit so as to produce an epilepsy phenotype.

2. An isolated DNA molecule as claimed in claim 1 wherein said mutation event is a point mutation.

3. An isolated:DNA molecule as claimed in claim 2 wherein said point mutation results in substitution of a valine residue.

4. An isolated DNA molecule as claimed in claim 3 wherein said point mutation results in a valine residue being substituted by an amino acid having a more bulky side chain and/or a β-carbon atom substituted only by hydrogen atoms.

5. An isolated DNA molecule as claimed in claim 4 wherein said point mutation results in a valine residue being replaced by methionine or leucine.

6. An isolated DNA molecule as claimed in claim 5 wherein said point mutation results in replacement of V287 in the β2 subunit.

7. An isolated DNA molecule as claimed in claim 6 wherein said point mutation is a G to A nucleotide transition at base 1025 in order to produce a V287M transition in the β2 subunit.

8. An isolated DNA molecule as claimed in claim 7 wherein the DNA molecule comprises the nucleotide sequence set forth in SEQ ID NO:1.

9. An isolated DNA molecule as claimed in any one of claims 1 to 7 in which one or more additional mutation events selected from the group consisting of point mutations, deletions, insertions and rearrangements have occurred.

10. An isolated DNA molecule as claimed in claim 9 wherein said one or more additional mutation events are point mutations which result in conservative amino acid substitutions within the β-subunit.

11. An isolated DNA molecule comprising the nucleotide sequence set forth in SEQ ID NO:1.

12. An isolated DNA molecule consisting of the nucleotide sequence set forth in SEQ ID NO:1.

13. An isolated polypeptide, said polypeptide being a mutant β-subunit of a mammalian nicotinic acetylcholine receptor (nAchR), wherein a mutation event selected from the group consisting of substitutions, deletions, insertions and rearrangements has occurred in the M2 domain and said mutation event disrupts the functioning of an assembled mammalian nicotinic acetylcholine receptor so as to produce an epilepsy phenotype.

14. An isolated polypeptide as claimed in claim 13 wherein said mutation event is a substitution.

15. An isolated polypeptide as claimed in claim 14 wherein there is substitution of a valine residue.

16. An isolated polypeptide as claimed in claim 15 wherein said valine residue is substituted by an amino acid having a more bulky side chain and/or a β-carbon atom substituted only by hydrogen atoms.

17. An isolated polypeptide as claimed in claim 16 wherein said valine residue is replaced by methionine or leucine.

18. An isolated polypeptide as claimed in claim 17 wherein said valine is V287 in the β2 subunit.

19. An isolated polypeptide as claimed in claim 18 wherein the substitution is a V287M transition in the β2 subunit.

20. An isolated polypeptide as claimed in claim 19 comprising the amino acid sequence set forth in SEQ ID NO:2.

21. An isolated polypeptide as claimed in claims 13 to 19 in which one or more additional mutation events selected from the group consisting of substitutions, deletions, insertions and rearrangements have occurred.

22. An isolated polypeptide as claimed in claim 21 wherein said one or more mutation events are conservative substitutions.

23. An isolated polypeptide comprising the amino acid sequence set forth in SEQ ID NO:2.

24. An isolated polypeptide consisting of the amino acid sequence set forth in SEQ ID NO:2.

25. An isolated polypeptide, said polypeptide being an assembled mammalian nicotinic acetylcholine receptor, comprising at least one α-subunit and at least one β-subunit, wherein a mutation event selected from the group consisting of substitutions, deletions, insertions and rearrangements has occurred in the M2 domain of a β-subunit and said mutation event disrupts the functioning of said assembled mammalian nicotinic acetylcholine receptor so as to produce an epilepsy phenotype.

26. An isolated polypeptide as claimed in claim 25 wherein said mutation event is a substitution.

27. An isolated polypeptide as claimed in claim 26 wherein there is substitution of a valine residue.

28. An isolated polypeptide as claimed in claim 27 wherein said valine residue is substituted by an amino acid having a more bulky side chain and/or a β-carbon atom substituted only by hydrogen atoms.

29. An isolated polypeptide as claimed in claim 28 wherein said valine residue is replaced by methionine or leucine.

30. An isolated polypeptide as claimed in claim 29 wherein said valine is V287 in the β2 subunit.

31. An isolated polypeptide as claimed in claim 30 wherein the substitution is a V287M transition in the β2 subunit.

32. An isolated polypeptide as claimed in claim 31 wherein the β2 subunit comprises the amino acid sequence set forth in SEQ ID NO:2.

33. An isolated polypeptide as claimed in claims 25 to 32 in which one or more additional mutation events selected from the group consisting of substitutions, deletions, insertions and rearrangements have occurred.

34. An isolated polypeptide as claimed in claim 33 wherein said one or more mutation events are conservative substitutions.

35. An isolated polypeptide as claimed in any one of claims 25 to 34 wherein a further mutation event selected from the group consisting of substitutions, deletions, insertions and rearrangements has occurred in the M2 domain of at least one further β-subunit, and said further mutation event independently disrupts the functioning of said assembled mammalian nicotinic acetylcholine receptor so as to produce an epilepsy phenotype.

36. An isolated polypeptide as claimed in claim 35 wherein said assembled nicotinic acetylcholine receptor comprises a plurality of β2 subunits, and an identical mutation event has occurred in any one or all of these.

37. An isolated polypeptide as claimed in claim 36 wherein said assembled nicotinic acetylcholine receptor consists of three β2 subunits with a V287M mutation in any one or all of these, and two α4 subunits.

38. A method preparing a polypeptide, said polypeptide being a mutant β-subunit of a mammalian nicotinic acetylcholine receptor, comprising the steps of:
(1) culturing host cells transfected with an expression vector comprising a DNA molecule as claimed in any one of claims 1 to 12 under conditions effective for polypeptide productions; and
(2) harvesting the mutant β-subunit.

39. A method as claimed in claim 38 further comprising the step of allowing the mutant β-subunit and other subunits of the mammalian nicotinic acetylcholine receptor to assemble into a mammalian nicotinic acetylcholine receptor and harvesting the assembled receptor.

40. An antibody which is immunologically reactive with a polypeptide as defined in any one of claims 13 to 37, but not with a wild-type nicotinic acetylcholine receptor or subunit thereof.

41. An antibody as claimed in claim 40 which is a monoclonal antibody.

42. A method of treating epilepsy, comprising administering a selective antagonist of the nicotinic acetylcholine receptor when it contains a mutation in the M2 domain of a β-subunit, said mutation being causative of epilepsy, to a subject in need of such treatment.

43. A method as claimed in claim 42 wherein the selective antagonist is an antibody.

44. A method as claimed in claim 43 wherein the antibody is a monoclonal antibody.

45. A method as claimed in any one of claims 42 to 44, further comprising the step of introducing a wild-type nicotinic acetylcholine receptor to said subject.

46. A method as claimed in claim 45 wherein the wild-type nicotinic acetylcholine receptor is introduced by gene therapy.

47. A method as claimed in claim 45 wherein the wild-type nicotinic acetylcholine receptor is introduced by administering a substantially purified wild-type nicotinic acetylcholine receptor or nicotinic acetylcholine receptor β-subunit polypeptide.

48. The use of a selective antagonist of the nicotinic acetylcholine receptor when it contains a mutation in the M2 domain of a β-subunit, said mutation being causative of epilepsy, in the manufacture of a medicament for the treatment of epilepsy.

49. A method of treating epilepsy, comprising administering an isolated DNA molecule which is the complement of any one of the DNA molecules defined in claims 1 to 12 and which encodes a mRNA that hybridises with the mRNA encoding the β-subunit of the nicotinic acetylcholine receptor when it contains a mutation causative of epilepsy in the M2 domain, to a subject in need of such treatment.

50. A method as claimed in claim 49, further comprising the step of introducing a wild-type nicotinic acetylcholine receptor to said subject.

51. The use of an isolated DNA molecule which is a complement of a DNA molecule as defined in any one of claims 1 to 12 and which encodes a mRNA that hybridises with the mRNA encoding the β-subunit of the nicotinic acetylcholine receptor when it contains a mutation causative of epilepsy in the M2 domain, in the manufacture of a medicament for the treatment of epilepsy.

52. The use of an isolated DNA molecule as claimed in any one of claims 1 to 12 for the diagnosis of epilepsy.

53. The use of a polypeptide as defined in any one of claims 13 to 37 in the diagnosis of epilepsy.

54. The use of an antibody as claimed in either claim 40 or 41 in the diagnosis of epilepsy.

55. A method for the diagnosis of epilepsy, comprising the steps of:
(1) obtaining DNA from a subject suspected of epilepsy; and
(2) comparing the DNA sequence of a β-subunit of the nicotinic acetylcholine receptor of said DNA to the DNA sequence of the corresponding β-subunit of the wild-type nicotinic acetylcholine receptor.

56. A method as claimed in claim 55 wherein each DNA fragment is sequenced and the sequences compared.

57. A method as claimed in claim 55 wherein the DNA fragments are subjected to restriction enzyme analysis.

58. A method as claimed in claim 55 wherein the DNA fragments are subjected to SSCP analysis.

59. A method for the diagnosis of epilepsy, comprising the steps of:
(1) obtaining the nicotinic acetylcholine receptor from a subject suspected of epilepsy; and
(2) comparing a β-subunit of said receptor with the corresponding β-subunit of the wild-type nicotinic acetylcholine receptor.

60. Use of a polypeptide as defined in any one of claims 13 to 37 in the screening of candidate pharmaceutical agents.

61. Use as claimed in claim 60 wherein high-throughput screening techniques are employed.

62. A genetically modified non-human animal transformed with an isolated DNA molecule as defined in any one of claims 1 to 12.

63. A genetically modified non-human animal as claimed in claim 62 in which the animal is selected from the group consisting of rats, mice, hamsters, guinea pigs, rabbits, dogs, cats, goats, sheep, pigs and non-human primates such as monkeys and chimpanzees.

64. A genetically modified non-human animal as claimed in claim 63 wherein the animal is a mouse.

65. The use of a genetically modified non-human animal as claimed in any one of claims 62 to 64 in the screening of candidate pharmaceutical compounds.

66. The use of a cell transformed with a DNA molecule as claimed in any one of claims 1 to 12 in the screening of candidate pharmaceuticals.

67. A host cell transformed with a DNA molecule as claimed in any one of claims 1 to 12.

68. An expression vector comprising a DNA molecule as claimed in any one of claims 1 to 12.
